(19) United States
(12) Patent Application Publication
(10) Pub. No.: US 2019/0110985 A1
(43) Pub. Date: Apr. 18, 2019

(54) Process for preparing shaped articles for administration to animals

(71) Applicant: Bayer Animal Health GmbH, Leverkusen (DE)

(72) Inventors: Venkata-Rangarao KANIKANTI, Leverkusen (DE); Hans-Juergen HAMANN, Dormagen (DE); Peter KLEINEBUDDE, Duesseldorf (DE); Carmen STOMBERG, Rheinberg (DE)

(21) Appl. No.: 16/099,467

(22) PCT Filed: May 5, 2017
(86) PCT No.: PCT/EP2017/060805

§ 371 Date: Nov. 7, 2018

(30) Foreign Application Priority Data
May 12, 2016 (EP) 16169374.2

(51) Int. Cl. A61K 9/00; A61K 9/14; A61K 45/06; A61P 33/10

(52) U.S. Cl. CPC A61K 9/0056; A61K 9/143; A61P 33/10; A61K 9/146; A61K 45/06; A61K 9/145

(57) ABSTRACT

The present invention relates to a process for preparing shaped articles for administration to animals, to the shaped articles obtainable according to the process and to their use.

The invention relates to a process for preparing shaped articles for administration to animals, to the shaped articles obtained therefrom and to the administration of the shaped articles to animals. The shaped articles serve in particular for the administration of pharmaceutically active substances.

### PRIOR ART

The acceptance of a pharmaceutical by the pet is determined mainly by its odour and taste (Thombre, A. G., 2004, Advanced Drug Delivery Reviews 56 (10), 1399-1413). Thus, for example, the use of flavourings leads to an increase in acceptance of more than 90% for bitter medicaments (Ahmed, I. and Kasraian, K. 2002, Advanced Drug Delivery Reviews 54 (6), 871-882). Cats favour the taste of fish, whereas dogs prefer that of beef, pork and lamb (Houpt, K. A. and Smith, S. L. 1981, Canadian Veterinary Journal 22(4), 77-85). The fact that, in spite of the high proportion of flavourings and taste improvers (Rose et al. 2008, U.S. Pat. No. 7,348,027) or in some cases also animal byproducts (Cleverly et al. 2004, WO2004016252), veterinary medicinal products are still rejected is due to the obvious discriminability to the food. In general, oral presentation forms are harder than conventional animal food or differ therefrom in their form.

The palatability of a chewable medicinal product is largely determined by the mouthfeel generated (Thombre, A. G., 2004, Advanced Drug Delivery Reviews 56 (10), 1399-1413). To generate a pleasant mouthfeel, the textures of the pharmaceutical are adapted to the preferences of the patients. In the case of dogs, soft or creamy structures are preferred considerably (Rose et al. 2008, U.S. Pat. No. 7,348,027) to hard and brittle structures, for example DE69937780 (Damon et al. 2008).

The preparation of meat jelly is described in: Anonymous: “Sülze/Sauerfleisch (Rezept mit Bild) von hawk69|Chefkoch.de”, 20 Apr. 2004 (2004-04-20), XP055313684, found on the internet: URLhttp://www.chefkoch.de/rezepte/190821081252760/Suelze-Sauerfleisch.html. Anonymous: “Dogranch Erftstadt Rezepte Hundesülze”, 25 Mar. 2009 (2009-03-25), XP055313791, found on the internet: URL: http://dogranch.homepage.t-online de/Rezept Suelze.htm describes dogfood in the form of meat jelly. Shaped articles prepared by extrusion for the administration of pharmaceutically active substances are not disclosed in these documents. What has been developed so far are chewables based on flavoured starch extrudates. The chewables are intended for mixing with the animal food employed, without the animal then being able to select (Isele 2008, AU2008201605 B2; Kalbe 2008, EP1296655 B1). By virtue of the high proportion of flavourings, the palatability is very good; however, this cannot be taken as granted for the entire storage period. Since starch-containing chewables get harder over time, as time passes, the mouthfeel generated is also perceived as more crumbly (Keetels, C. J. A. M. et al. 1996, Food Hydrocolloids 10 (3), 343-353).
A further disadvantage of the starch-containing chewables is the fact that it is not possible to embed thermolabile active compounds. This also applies to chewables produced from a sugar mixture heated under high pressure (Han, Y. D. and Park, J. B. 2002, U.S. Pat. No. 6,444,218; Han, Y. D. and Park, J. B. 2002, U.S. Pat. No. 6,440,450).

By retrogradation of starch-containing products, previously bound water is released again, resulting in a significant change of the physical structure (Farhat et al. 2001, Starch/Starke 53 (9), 431-436) and thus also the elasticity (Vandeputte et al. 2003. Journal of Cereal Science, 38 (19, 61-68) of the extrudates. To avoid these problems, very complicated recipes were developed which, in addition to the starch, contain a vegetable oil, sugar and further additives (Huron, S. 2004, WO2004/014143 A1; Paulsen et al. 2011, U.S. Pat. No. 7,955,632 B2). However, the oil, which is intended to improve the mouthfeel, causes problems during further processing of the chewables. Adhesion to tabletting tools or forming machines (Carrillo, B. and Freehauf, K. 2013, WO2013068371 A1) have been described. Moreover, during storage the oil is separated from the remainder of the formulation, so that these chewables, too, harden and the mouthfeel gets chalky.

In an attempt to prepare chewables of unchanging consistency and attractiveness, other inventions dispensed with starch (Gao et al. 2010, US20100291245; Hamann, H. J. and Kanikanti, V.-R. 2012, WO2012049156 A1). However, owing to their great hardness, it is not possible to reconcile the texture of these chewables with the preferences of dogs. The texture is brittle and the mouthfeel is crumbly. In addition, the invention of Gao requires a drying process of several hours until the chewable has reached the desired moistness.

### OBJECT OF THE INVENTION

It was the object of the invention to provide a process for preparing shaped articles for administration to animals, in particular dogs, and the shaped articles obtained therefrom, in particular for the administration of pharmaceutically active substances. In particular, it was the object of the present invention to provide a chewable medicinal product (hereinbelow occasionally referred to as chewables or soft chewables) which stands out from the known products in particular in the following points:

1) High palatability is to be achieved by the chewable medicinal product having a pleasant taste and a texture profile well-accepted by dogs, ensured by the following characteristics:

a. no breaking of the chewables at a certain force load,

b. sufficient elasticity and plasticity at a certain force load,

c. the dog should chew the chewable about as often as a piece of meat sausage (on average at least 3 times).

2) The chewable medicinal product has a pleasant mouthfeel. Chewing should lead neither to a chalky nor to a crumbly feel.

3) Chewing of the medicinal product is optional: release from unchewed pieces should not be delayed compared to release from comparison tablets.

4) The disintegration time of the chewable medicinal product according to Ph. Eur. 8.0 should be below 30 min.

5) The storage time should have a negligible effect on the properties of the medicinal product, for example

a. the disintegration time should stay below 30 min,

b. the release properties should not deteriorate,

c. the pieces formed in the process of the present invention should not harden but remain elastic and firm to the bite.

6) The preparation process must meet the following requirements:

a. Robustness,

b. Option of a continuous process,

c. No adhesion to process parts,

d. Curing not required,

e. Can be carried out at temperatures below 50° C., ideally at room temperature.

7) Preferably, the recipe should contain neither starch nor sucrose.

Meeting these extensive and demanding requirements with respect to the properties of the process and the corresponding shaped articles for the administration of pharmaceutically active substances to animals can only be achieved, surprisingly, by combining an ingredient composition developed for this purpose and a production process likewise developed for this purpose.

### DESCRIPTION OF THE INVENTION

The invention thus provides a process for preparing shaped articles for administration to animals, which process comprises the following steps:
a) Provision of a powder mixture of solid ingredients of the shaped articles,
b) Mixing of the powder mixture obtained in step a) with at least one first liquid F_{V} having a volume F1, giving a preswollen mixture,
c) Mixing of the preswollen mixture obtained in step b) with at least one second liquid F_{K} having a volume F2, where F2>F1, giving a swollen mixture,
d) Forming of the swollen mixture obtained in step c) with formation of the shaped articles.

For the purpose of the present invention, a shaped article is to be understood as an object obtained by a formative process, preferably by cutting a strand obtained by extrusion. The shaped articles preferably have a smooth surface, no tears visible to the naked eye and no irregularities that can be felt by hand, and a volume of up to 10 ml.

The shaped articles produced by the process of the present invention are for administration to animals, and they can be employed in animal husbandry and animal breeding for farm animals, breeding animals, zoo animals, laboratory animals, research animals and pets, and in particular for mammals.

The farm animals and breeding animals include mammals such as, for example, cattle, horses, sheep, pigs, goats, camels, water buffalos, donkeys, rabbits, fallow deer, reindeer, fur-bearing animals, such as, for example, minks, chinchilla, racoon, and also birds such as, for example, chicken, geese, turkeys, ducks, pigeons and ostriches. Examples of preferred farm animals are cattle, sheep, pig and chicken.

Laboratory animals and research animals include dogs, cats, rabbits and rodents such as mice, rats, guinea pigs and golden hamsters.

The pets include dogs, cats, horses, rabbits, rodents such as golden hamsters, guinea pigs, mice, furthermore reptiles, amphibians and birds that are kept at home or in a zoo. Administration to cats and dogs is particularly preferred.

Since the shaped articles according to the invention are administered to animals orally and, during administration, are usually chewed, when reference is made to the shaped articles according to the invention the term “chewable” or “chewables” is also used.

Providing the powder mixture comprises mixing the solid ingredients, that is the solid pulverulent components, defined below, of the shaped articles, for example in a mixer, a kneader or an extruder, most preferably an extruder.

The powder mixture provided does not have to comprise the entire amount of the solid ingredients of the shaped articles, since part of the ingredients may also be introduced in the form of a solution via the preswelling and swelling liquids F_{V} and F_{K} described below.

Metered addition of the powder mixture obtained, for mixing with the liquid F_{V}, may take place, for example, using a powder feeder such as a gravimetric powder feeder.

Metered addition of the volume F1 of the liquid F_{V} may be by use of a suitable pump. Mixing of the liquid Fv, defined below, and the powder mixture gives a preswollen mixture, where the preswelling step is preferably carried out in a mixer, a kneader or an extruder, most preferably in an extruder.

Mixing of the preswollen mixture with a volume F2 of the second liquid F_{K}, defined below, gives a swollen mixture. Metered addition of the volume F2, which is always greater than the volume of the first liquid F1, may also be by use of a suitable pump. The swelling step is preferably carried out in a mixer, a kneader or an extruder, most preferably in an extruder.

Forming of the swollen mixture obtained as described gives the shaped articles, which are the subject of the invention. This forming step preferably takes place by cutting a strand, particularly preferably by cutting a strand obtained by extrusion.

In a preferred embodiment of the process, the ratio by volume of the volume of the second liquid F2 to the volume of the first liquid F1 is >1.1 and is in the range from 1.2 to 5, even better in the range from 1.5 to 3, if the process is carried out in an extruder.

If the process is carried out in a kneader, the ratio of F2 to F1 is preferably in the range from 2 to 100, more preferably in the range from 20 to 80, most preferably in the range from 30 to 50.

In a preferred embodiment of the process, the ratio by weight of the amount of the first liquid F_{V} to the amount of the solid ingredients in the powder mixture when carrying out the process in an extruder is in the range from 0.01 to 1, particularly preferably in the range from 0.1 to 0.8, very particularly preferably in the range from 0.3 to 0.7.

In a preferred embodiment of the process, the ratio by weight of the amount of the first liquid F_{V} to the amount of the solid ingredients in the powder mixture when carrying out the process in a kneader is in the range from 0.005 to 0.1, particularly preferably in the range from 0.01 to 0.05, very particularly preferably in the range from 0.02 to 0.03.

In a preferred embodiment of the process, the ratio by weight of the amount of the second liquid F_{K} to the amount of the solid ingredients in the powder mixture when carrying out the process is in the range from 0.1 to 2, particularly preferably in the range from 0.2 to 1.5, and particularly preferably in the range from 0.6 to 1.3.

In preferred embodiments of the process, the liquids F_{V} and F_{K} independently of one another consist of water, glycerol or mixtures of water and glycerol. The consistency of the shaped articles according to the invention can be adjusted as desired by using a gel former, as defined below, together with the chosen liquids F_{V} and F_{K}. With glycerol, a hydrophilic liquid component having a low vapour pressure was chosen for combination with the gel former, of which the gel former can physically bind its own weight many times over. In binding to the gel former, glycerol acts as plasticizer for the preparation and is at the same time part of the consistency-conferring system. In addition, glycerol can, according to the European Food Safety Authority (EFSA), be used in animal food without any restriction and is listed as “GRAS” (=“generally recognized as safe”, Handbook of Pharmaceutical Excipients, Eds. Wade, Weller, 2^{nd} Ed. (1994), American Pharmaceutical Association, Washington, p. 204).

In preferred embodiments of the process according to the invention, the shaped articles obtained after step d) contain 5 to 40% by weight of glycerol, particularly preferably 10 to 35% by weight of glycerol.

In a further preferred embodiment of the process according to the invention, the shaped articles according to the invention contain 5-20% by weight of water, for example 10-20% by weight of water. If the shaped articles are to contain little water, the water content is preferably at 5-10% by weight of water.

In addition, the first liquid F_{V} and the second liquid F_{K} of the claimed process may independently of one another each comprise one or more ingredients, which may be either pharmaceutically active ingredients or other ingredients, for example gel formers or auxiliaries, which can be introduced, by prior dissolution in the liquid F_{V} or F_{K}, as dissolved components into the mixture forming the shaped articles.

In a preferred embodiment of the process according to the invention, the first liquid F_{V} of the process contains in each case one or more ingredients, which may be either pharmaceutically active ingredients or other ingredients such as gel formers or auxiliaries, which can be introduced in form of a solution in liquid F_{V} into the mixture forming the shaped articles.

In an even more preferred embodiment of the process according to the invention, the one or more ingredients introduced in the liquid are humectants such as glycerol, propylene glycol and hyaluronic acid.

In a particularly preferred embodiment of the process according to the invention, one of the humectants contained in the first liquid F_{V} of the process is hyaluronic acid which very particularly preferably has a number-average molecular weight of from 8000 to 15 000. By adding a small amount of this additional hydrogel former at the start of the kneading process, it is possible to reduce the swelling time and thus the time of the entire process markedly.

In a preferred embodiment of the process according to the invention, the shaped articles obtained after step d) contain 0.001 to 5% by weight hyaluronic acid, particularly preferably 0.01 to 3% by weight hyaluronic acid, and very particularly preferably 0.01 to 1.0% by weight hyaluronic acid.

In a preferred embodiment of the process, the period of time between the addition of the preswelling liquid F_{V} in step b) and the swelling liquid F_{K} in step c), referred to as preswelling time, is up to 300 s.

The total processing time required is reduced when the preswelling time is increased, until an optimum is reached. Once this optimum preswelling time is exceeded, the processing time increases again.

In a preferred embodiment of the process according to the invention, where the process is carried out in a kneader, the processing time between the addition of the preswelling liquid F_{V} and the forming step d), that is the sum of preswelling time and swelling time, is preferably from 30 to 120 minutes, particularly preferably from 40 to 90 minutes.

In a preferred embodiment of the process according to the invention, where the process is carried out in an extruder, the processing time between the addition of the preswelling liquid F_{V} and the forming step d), that is the sum of preswelling time and swelling time, is preferably from 1 to 6 minutes, particularly preferably from 2 to 5 minutes.

Besides the formulation, the total time depends on the temperature, the preswelling time, the type of mixing device, the desired consistency and the set pH of the mixture used.

In a preferred embodiment of the process according to the invention, the mixing temperature does not exceed 50° C., even more preferably not 40° C., and particularly preferably the mixing temperature is between 20 and 30° C. This also allows processing of thermosensitive compounds as active compounds in the shaped articles obtained in the process described.

In a preferred embodiment of the process according to the invention, where the process is carried out in an extruder, the optimum mixing temperature is in the range from 20 to 50° C., preferably 30 to 50° C.

Particularly suitable devices for carrying out the process according to the invention are mixers, kneaders and extruders, and the process is therefore preferably carried out in one of these devices. Particularly preferably, the process according to the invention is carried out in an extruder.

For the purpose of the invention, an extruder is to be understood as an apparatus which is used for extrusion, that is the continuous squeezing of solid to highly viscous materials under pressure from a formative opening, also referred to as die, matrix or nozzle. The resulting bodies, having a cross section which corresponds to the opening, and of theoretically any length, are accordingly referred to as extrudates.

Mixing of the solid powder components takes place after charging of the extruders with the solid components via an inlet opening, for example via an inlet funnel. Further components of the extrusion mixture may also be introduced into the extruder via other inlet openings at different locations. Homogenization and plastification during transport through the frequently stepped screw in the extruder may take place with heating or cooling, until finally the material is squeezed through the formative die in the spray head of the extruder.

A characteristic distinguishing feature of an extruder is the ratio L/D of length L to diameter D of the extruder screw.

In a preferred embodiment of the process, the L/D ratio is in the range of 35-45.

For the purpose of the invention, the term extruder comprises mono- and multi-screw extruders, planetary roller extruders and cascade extruders, without being limited to these extruder variants.

In a preferred embodiment, steps a) to c) of the process according to the invention are carried out continuously in an extruder.

In a preferred embodiment, the forming in step d) comprises the cutting of a strand.

In a preferred embodiment of the process according to the invention, this does not include a subsequent hardening or curing step of the shaped articles after the formative step d). The shaped articles obtained in step d) have the consistency desired for the application and the appropriate moisture content and can therefore be packaged and/or stored protected from moisture immediately after step d).

In a preferred embodiment of the process according to the invention, the ingredients of the shaped articles are selected from the group consisting of (a) one or more pharmaceutically active substances, (b) one or more flavourings, (c) one or more gel formers, (d) one or more fillers, (e) one or more liquids selected from the group of the liquids F_{V} and F_{K} and (f) optionally one or more auxiliaries such as formulation auxiliaries, glidants, lubricants, disintegrants, humectants and preservatives.

Pharmaceutically active substances which may be contained in the shaped articles obtained in the process according to the invention are, in principle, all possible active compounds which are commonly administered orally to animals.

The active compounds comprise, for example, antimicrobially active compounds, such as antiviral active compounds, antibiotic active compounds and those acting against protozoa, such as coccidia; furthermore, for example, anti-inflammatory and psychotropic active compounds and also proton pump inhibitors etc., and also particularly preferably active compounds acting against parasites (ectoparasites and/or endoparasites), such as acaricidal, insecticidal, anthelmintic active compounds.

In a preferred embodiment of the process according to the invention, the pharmaceutically active substance(s) a) are antimicrobial substances or antibiotics, preferably for bacterial disorders, and antiparasitics.

In another preferred embodiment of the process according to the invention, the pharmaceutically active substance(s) a) are active compounds against coccidia.

In a particularly preferred embodiment of the process according to the invention, the antiparasitics are ectoparasiticides, in particular arthropodicides, namely insecticides and acaricides, and endoparasiticides, in particular anthelminthics.

In a very particularly preferred embodiment of the process according to the invention, the antiparasitics are insecticides, acaricides and anthelminthics.

Examples of suitable active compounds are the following known classes: acaricides, such as the macrocycles abamectin, doramectin, eprinomectin, ivermectin, milbemectin, nikkomycins, selamectin, tetranactin and thuringiensin; bridged diphenyl acaricides such as azobenzene, benzoximate, benzyl benzoate, bromopropylate, chlorbenside, chlorfenethol, chlorfenson, chlorfensulphide, chlorobenzilate, chloropropylate, dicofol, diphenyl sulfone, dofenapyn, fenson, fentrifanil, fluorbenside, proclonol, tetradifon and tetrasul; carbamate acaricides such as benomyl, carbanolate, carbaryl, carbofuran, fenothiocarb, methiocarb, metolcarb, promacyl and propoxur; oxime carbamate acaricides such as aldicarb, butocarboxim, oxamyl, thiocarboxim and thiofanox; dinitrophenol acaricides such as binapacryl, dinex, dinobuton, dinocap, dinocap-4, dinocap-6, dinocton, dinopenton, dinosulfon, dinoterbon and DNOC; formamidine acaricides such as amitraz, chlordimeform, chloromebuform, formetanate and formparanate; growth regulators for mites such as clofentezine, dofenapyn, fluazuron, flubenzimine, flucycloxuron, flufenoxuron and hexythiazox; organochlorine acaricides such as bromocyclen, camphechlor, dienochlor and endosulfan; pyrazole acaricides such as acetoprole, fipronil and analogues and derivatives thereof, tebufenpyrad, pyriprole and vaniliprole; pyrethroid acaricides such as, for example, pyrethroid ester acaricides such as acrinathrin, bifenthrin, cyhalothrin, cypermethrin, alpha-cypermethrin, fenpropathrin, fenvalerate, flucythrinate, flumethrin, fluvalinate, tau-fluvalinate and permethrin, pyrethroid ether acaricides such as halfenprox; quinoxaline acaricides such as quinomethionate and thioquinox; sulfite ester acaricides such as propargite; tetronic acid acaricides such as spirodiclofen; and acaricides not belonging to a particular class, such as ace-quinocyl, amidoflumet, arsenic oxide, chlormethiuron, closantel, crotamiton, diafenthiuron, dichlofluanid, disulfiram, fenazaflor, fenazaquin, fenpyroximate, fluacrypyrim, fluenetil, mesulfen, mnaf, nifluridide, pyridaben, pyrimidifen, sulfiram, sulfluramid, sulfur and triarathene.

Insecticides may belong to various chemical classes, such as, for example, chlorinated hydrocarbons, organophosphates, carbamates, pyrethroids, formamidines, borates, phenylpyrazoles and macrocyclic lactones. Known insecticides include imidacloprid, fenthion, fipronil, allethrin, resmethrin, fenvalerate, permethrin, malathion and derivatives thereof. According to one embodiment, insecticides of the neonicotinoid class are preferred, for example acetamiprid, clothianidin, dinotefuran, imidacloprid (see above), nitenpyram, thiacloprid and thiamethoxam. Frequently used growth-regulating active compounds (insect growth regulators, IGRs) are, for example, benzoylphenyl ureas, such as diflubenzuron, lufenuron, noviflumuron, hexaflumuron, triflumuron and teflubenzuron or active ingredients such as fenoxycarb, pyriproxyfen, methoprene, kinoprene, hydroprene, cyromazine, buprofezin, pymetrozine and derivatives thereof.

Anthelmintics may be endoparasiticides or endectocides and encompass the following well-known groups: macrocyclic lactones, benzimidazoles, probenzimidazoles, imidazothiazoles, tetrahydropyrimidines, organophosphates, piperazines, salicylanilides and cyclic depsipeptides (see below).

Preferred anthelmintics encompass macrocyclic lactones having a broad spectrum, such as avermectins, milbemycins and derivatives thereof, such as, for example, ivermectin, doramectin, moxidectin, selamectin, emamectin, eprinomectin, milbemectin, abamectin, milbemycin oxime, nemadectin and derivatives thereof. The classes of the benzimidazoles, benzimidazole carbamates and probenzimidazoles also encompass active compounds such as thiabendazole, mebendazole, fenbendazole, oxfendazole, oxibendazole, albendazole, luxabendazole, netobimin, parbendazole, flubendazole, cyclobendazole, febantel, thiophanate and derivatives thereof. Imidazothiazoles encompass active compounds such as tetramisole, levamisole and derivatives thereof. The tetrahydropyrimidines encompass active compounds such as morantel, pyrantel and derivatives thereof. Organophosphates encompass active compounds such as dichlorvos, haloxon, trichlorfon and derivatives thereof. Salicylanilides encompass active compounds such as closantel, tribromsalan, dibromsalan, oxyclozanide, clioxanide, rafoxanide, brotianide, bromoxanide and derivatives thereof. Cyclic depsipeptides encompass compounds having 6 to 30 ring atoms and are composed of amino acids and hydroxycarboxylic acids as structural units of the ring.

Antimicrobial compounds are, for example, various penicillins, tetracyclines, sulfonamides, cephalosporins, cephamycin, aminoglucosides, trimethoprim, dimetridazole, erythromycin, framycetin, fruazolidone, various pleuromutilins such as tiamulin, valnemulin, various macrolides, streptomycin, clopidol, salinomycin, monensin, halofuginone, narasin, robenidine, quinolones, etc. Specific examples of fluoroquinolones include benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, perfloxacin, temafloxacin, tosufloxacin, sarafloxacin and sparfloxacin. A further example of an antibacterial fluoroquinolone that can be used in animals is pradofloxacin. Specific examples of other quinolones include pipemidic acid and nalidixic acid.

Apart from the above-mentioned active pharmaceutical compounds, it is also possible to have vitamins or minerals, for example, as constituents.

The active ingredients may preferably be, for example, depsipeptides selected from the group consisting of PF 1022A and emodepside.

Preferred antimicrobial fluoroquinolones are in particular enrofloxacin or pradofloxacin.

In a particularly preferred embodiment, the shaped articles according to the invention contain an active ingredient selected from febantel, pyrantel (typically in the form of a salt, the embonate being preferred) and praziquantel or a two-part combination composed of said active ingredients. Even more preferably, febantel, pyrantel embonate and praziquantel are used as a three-part combination in the shaped articles according to the invention.

The active compounds can also—where applicable—be used in the form of their salts with pharmaceutically acceptable acids or bases or else as solvates, more particularly hydrates, of the active compounds or their salts.

Prodrugs of the active compounds can also be used.

In a preferred embodiment of the process according to the invention, the solid active compounds used as ingredients a) are present at a mean particle size D(50) of 100 nm to 10 μm, preferably 100 nm to 5 μm.

For the purpose of the present invention, D(50) is understood as a volume-based particle size distribution at which 50% of all particles have a dimension (diameter) smaller than or equal to this value. The particle sizes indicated here are determined by the laser diffraction method using the Mastersizer 2000 apparatus (Hydro 2000G dispersion unit) from Malvern and the Fraunhofer diffraction evaluation mode, since the refractory indices of the active compound particles are unknown. Here, with stirring, a suitable amount of the sample is predispersed with 2-3 ml of a dispersing medium (low-viscosity paraffin). The dispersion is then introduced into the dispersion unit of the apparatus and measured. Depending on the setting, the evaluation software states the particle size as D(50) values, D(10) values, D(90) values, etc.

According to the invention the shaped articles contain at least one active compound in a pharmaceutically effective amount, where “pharmaceutically effective amount” represents a non-toxic amount of active compound which can cause the desired effect. The amount of active compound employed depends on the active compound, the treated animal and the nature, the severity and the stage of the disease.

The shaped articles obtained by the process according to the invention contain one or more flavourings, preferably meat flavourings. Meat flavouring refers to an additive which is of synthetic or animal origin or a mixture of the two and which confers a meat-like odour and/or taste to the shaped articles obtained in the described process according to the invention. Particularly preferably, use is made of meat flavourings purely of animal origin. These are prepared, for example, from beef, poultry, fish, animal skins or animal livers. Even more preference is given to desiccated liver powder, for example from cattle, sheep, poultry or pig and very particularly preferably from poultry or pig.

As already mentioned, the shaped articles prepared by the process according to the invention contain one or more gel formers. Such a gel former is characterized, inter alia, by its ability to bind glycerol physically. This property is of fundamental importance for obtaining the desired consistency of the shaped articles. The ability of a gel former to bind glycerol physically is described by the glycerol binding value (hereinbelow referred to as GBV). Experimentally, this value is determined as follows:

100.00 mg of a gel former are weighed out into vessels with snap-on lid and suspended therein in 10.00 g of glycerol (85%) by shaking. After 24 h, in each case 1.000 g of the mixture is transferred to a polyether sulfone molecular sieve (Vivaspin 4 MOCW 5 kDa, Satorius Stedim Biotech GmbH, Gottingen, Germany). The molecular sieves are then centrifuged in the Multifuge 100 (Heraeus, Hanau, Germany) at 4500·g. After a fixed period of time, about 3 h, the weight above the molecular sieve is determined and the binding value is calculated using the following equation:

$$GBV = \frac{{m}_{a} - {m}_{b}}{{m}_{b}}$$

where mₐ=residue on the molecular sieve, m_{b}=original mass of the gel former in 1 g of suspension.

In a preferred embodiment of the process according to the invention, the selected gel former c) has a GBV of more than 40 after 3 hours, in a further preferred embodiment a GBV of more than 60 after 3 hours.

In a preferred embodiment of the process, the gel former(s) c) is/are selected from the group of compounds consisting of cellulose derivatives, polyacrylic acids, pectins, alginates, agar, carrageen, xanthan gum, poloxamers (e.g. under the trade name “Pluronics”, (polyoxyethylene/polyoxypropylene block copolymers) and high molecular weight macrogols.

In a preferred embodiment of the process for preparing shaped articles, no gelatine is used.

In a particularly preferred embodiment of the process, the cellulose derivative(s) used as gel formers c) is/are selected from the group consisting of carboxymethylcellulose, hydroxyethylcellulose, hydroxypropylcellulose, methylcellulose and ethylcellulose.

In a very particularly preferred embodiment, the cellulose derivative used as gel former c) is crosslinked carboxymethylcellulose (croscarmellose).

In a preferred embodiment of the process, the shaped articles obtained by the process described contain one or more cellulose derivatives and hyaluronic acid.

In a preferred embodiment of the process of the present invention, the fillers d) are selected from the group consisting of solid sugar alcohols and inorganic calcium, magnesium, sodium and potassium salts.

In a particularly preferred embodiment of the process, the sugar alcohol used as filler d) is selected from the group consisting of compounds of the formula:

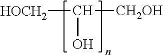

in which n≥3.

In a very particularly preferred embodiment of the process, the sugar alcohol used as filler d) is mannitol, xylitol or sorbitol.

In a further preferred embodiment of the process, at least one of the fillers used is selected from the group consisting of: lactose, cellulose, starch, sucrose and poorly soluble inorganic salts. Here, poorly soluble salts are salts whose solubility in 100 g of water at 20° C. is less than 1 g. Here, dicalcium phosphate dihydrate is particularly preferred.

In a preferred embodiment of the process, the shaped articles obtained contain less than 5% by weight starch or starch products.

Preference is also given to an embodiment of the process in which the shaped articles obtained contain less than 5% by weight sucrose.

Additionally, the shaped articles prepared by a preferred embodiment of the process may optionally contain one or more auxiliaries. Auxiliaries are preservatives, antioxidants and colourants.

Suitable auxiliaries and the required amounts to be employed are known in principle to the person skilled in the art. Suitable preservatives are, for example, sorbic acid, parabens, sodium propionate and sodium benzoate. Suitable antioxidants are, for example, butylated hydroxyanisol (BHA), butylated hydroxytoluene (BHT), propyl gallate, tocopherol, sodium metabisulfite or sodium ascorbate. Suitable colourants are organic and inorganic colourants or pigments suitable for pharmaceutical purposes, such as, for example, iron oxide. Other suitable auxiliaries are lubricants and glidants such as, for example, magnesium stearate, stearic acid, talc, bentonite or sodium stearylfumarate, disintegrants such as starch, crosslinked polyvinylpyrrolidone, croscarmellose-sodium (sodium salt of crosslinked carboxymethylcellulose), sodium starch glycolate (“Explotab®”), binders such as, for example, starch, low molecular weight cellulose ethers such as, for example, hydroxypropylcellulose and hydroxypropylmethylcellulose, sodium alginates or linear polyvinylpyrrolidone and also dry binders such as microcristalline cellulose and sugar alcohols. An exemplary selection of oils which can be used as auxiliaries comprises mineral oils, miglyols such as Miglyol 812 and Miglyol 814 (medium chain triglycerides and propylene glycol esters) and vegetable oils such as sunflower oil, soya bean oil, sesame oil, cottonseed oil, rapeseed oil, maize oil. It is also possible to use solubilizers as auxiliaries, for example oleoyl-macrogol-6-glycerides or glyceryl monolinoleate.

In a preferred embodiment, the shaped articles obtained by the process described above contain a) one or more pharmaceutically active substances, b) one or more flavourings, c) one or more gel formers, d) one or more fillers, e) one or more liquids selected from the group of the liquids F_{V} and F_{K}, and optionally one or more auxiliaries such as glidants, lubricants, disintegrants, humectants and preservatives.

In a preferred embodiment, in the shaped articles, the content of the pharmaceutically active substances a) is between 0.1 and 30% by weight, the content of the flavourings b) between 1 and 25% by weight, the content of the gel formers c) between 1 and 20% by weight, the content of the fillers d) between 5 and 60% by weight, preferably between 10 to 40% by weight, the content of the liquids e) F_{V} and/or F_{K} between 10 and 60% by weight, preferably between 15 and 40% by weight, and the content of one or more auxiliaries f) between 0 and 30% by weight, where the sum of the percentages is 100% by weight.

If the shaped articles contain compounds which can be assigned to two or more of groups a) to f), the maximum content of such a compound is defined by the sum of the respective weight ranges of the individual categories.

Preferred properties of the shaped articles prepared by the process described are a smooth surface without any tears visible to the naked eye or any irregularities that can be felt by hand, a volume of up to 10 ml, a disintegration time according to Ph. Eur. 8.0 of less than 30 minutes, a release according to Ph. Eur. 8.0 after 40 minutes of more than 50%, a moistness (loss on drying at 110° to a constant weight) of 3-30%, and a hardness determined by texture profile analysis (TPA) according to Bourne and Szczesniak (Bourne, M. C. and Szczesniak, A. S. 2003, “Sensory evaluation I Texture”, Encyclopedia of Food Sciences and Nutrition (Second Edition), B. Caballero, Oxford, Academic Press, 5167-5174) of between 2 and 15 N, particularly preferably between 2 and 7 N.

In a preferred embodiment, the shaped articles obtained are used for administration to animals, particularly preferably to dogs and cats, in the sense of a veterinary application.

In the field of animal health, i.e. the field of veterinary medicine, the shaped articles according to the invention can be employed against animal parasites, in particular ectoparasites or endoparasites. The term endoparasites comprises in particular helminths and protozoa such as coccidia. Ectoparasites are typically and preferably arthropods, in particular insects and acarids.

In the field of veterinary medicine, the shaped articles according to the invention are suitable for controlling parasites encountered in animal breeding and animal husbandry in farm animals, breeding animals, zoo animals, laboratory animals, research animals and pets.

By employing the active compounds according to the invention for controlling animal parasites, morbidity, mortality and reduced performance (for meat, milk, wool, hides, eggs, honey and the like) are to be reduced and/or prevented, so that more economical and simpler animal husbandry is possible and wellbeing of the animals can be improved.

With respect to the field of animal health, the term “control” or “controlling” means that the active compounds can effectively control the occurrence of the parasite in question in an animal infected by such a parasite to an extent which is harmless. More accurately, in the present context “controlling” means that the active compound can kill the parasite in question, prevent its growth or prevent its multiplication.

### I. Batchwise Production of Soft Chewables

### Example 1

Preparation of the chewables takes place in a measuring kneader (model W 50 EHT Brabender® GmbH & Co. KG, Duisburg, Germany). The volume of the kneading chamber is 55 cm³ minus the volume taken up by the blades used. For the preparations employed, this corresponds to a total weight of 70-85 g. For an effective kneading process, use is made of roller blades (W50, also Brabender®). 35 g of the powder components are mixed in the measuring kneader at 100 rpm at room temperature for 10 min. Here, the powder mixture has the following composition, where the sum of the ingredients excluding the pharmaceutical is 100% by weight and the pharmaceutical is praziquantel:

TABLE 1
Composition of the powder mixture, in % by weight

| | Proportion |
|---|---|
| | [%] |
| Pig liver powder | 24.3 |
| Dicalcium phosphate | 9.72 |
| Croscarmellose-Sodium | 17.36 |
| Mannitol | 48.61 |
| Pharmaceutical | q.s. |

The powder mixture remains in the kneader. The subsequent kneading process takes place at a rotational velocity of 50 rpm. The temperature of the kneading chamber is kept at 20° C. using a circulation thermostat Julabo F12 (Julabo Labortechnik GmbH, Seelbach, Germany).

At the start of the kneading process, 1 ml of the first liquid F_{V} (water with hyaluronic acid, 1 mg of HA/1 ml of water), which is used for preswelling, is added using a 1-ml syringe (BD Plastipak™ Becton Dickinson S.A., Madrid, Spain), after 30 seconds the second, consistency-conferring liquid F_{K} is injected into the kneading chamber in the form of 40 g of a 85% strength aqueous glycerol solution using a disposable 100-ml syringe (Omnifix® 100 ml, Braun, Melsungen, Germany). With increasing swelling of the material, the kneading process requires a higher force to be applied by the blades. The force is recorded as torque. Once a torque of 4 Nm is reached, the material has swollen sufficiently for removal and forming. After a processing time of 55 minutes, a consistency suitable for the formative step is reached. The finished kneading material is rolled to a plate of a thickness of 0.8 cm using a pizza machine (L30, Karl-Heinz Mussler GmbH, Heiligkreurtal, Germany) and cut into pieces of 1 cm·1 cm using a scalpel.

### Example 2

The process is carried out as in Example 1; however, the preswelling liquid contains no hyaluronic acid (F_{V}=1 ml of water) and the preswelling temperature is 30° C. The consistency suitable for the forming step, at which a torque of 4 Nm is recorded, is reached after a processing time of 26 minutes.

### Example 3

The process is carried out as in Example 2; however, the preswelling time is 180 seconds instead of 30 seconds. The consistency suitable for the forming step, at which a torque of 4 Nm is recorded, is reached after a processing time of 2⅓ minutes.

### Comparative Example 1

The process is carried out as described in Example 1; however, addition of the first liquid F_{V} is dispensed with and the second liquid F_{K} is added immediately, when the processing time starts. The consistency suitable for forming, at which a torque of 4 Nm is recorded, is reached after a processing time of 159 minutes.

### Comparative Example 2

The process is carried out as in Example 2; however, addition of the first liquid F_{V} is dispensed with and the second liquid F_{K} is added immediately, when the processing time starts. The consistency suitable for forming, at which a torque of 4 Nm is recorded, is reached after a processing time of 29 minutes.

### Comparative Example 3

The process is carried out as in Example 2; however, the preswelling time is 300 seconds instead of 30 seconds. The consistency suitable for the forming step, at which a torque of 4 Nm is recorded, is reached after a processing time of 11 minutes.

### II. Continuous Production of Soft Chewables

### Example 4

Continuous preparation of the shaped articles takes place in a twin-screw extruder (Pharmalab 16 twin-screw extruder, Thermo Fischer Scientific, Karlsruhe, Germany). Double liquid feeding ensures swelling inside the extruder. 1 kg of the powder components mentioned in Example 1 in Table 1 are, in the same composition, mixed in an LM20 laboratory mixer (Bohle, Eningerloh, Germany) for 15 min at 25 rpm and, before the extrusion is started, filled into a gravimetric powder feeder (K-CL-24-KT 20, K-Tron, Niederlenz, Switzerland). Prior to each process, the powder feeder is calibrated. The powder is added into the first cylinder of the extruder at a feeding rate of 10 g/min. Preswelling takes place between cylinder 2 and cylinder 6. In cylinders 6-10, swelling of the mixture occurs (see FIG. 2). The temperature of the mixture during the extrusion process is 25° C.

Stage 1 (preswelling): a peristaltic pump (Ismatec IPC 8/ISM 931, IDEX Health & Science GmbH, Wertheim, Germany) feeds 5 ml·min⁻¹ of the first liquid F_{V} (HA 1 mg/5 ml of water), which serves for preswelling, into the powder mixture in the second cylinder.

Stage 2 (swelling): 10 g·min⁻¹ 100% glycerol are fed into the sixth cylinder of the extruder using a micro annular gear pump (MZP 7205, HNP-Mikrosysteme, Schwerin, Germany) (see FIG. 2).

For the extrusion, a screw configuration having five different kneading zones is used to ensure a sufficiently long residence time and sufficient mixing. The screws have a diameter of 16 mm and a length of 41 D (41 times the diameter of the screw). At a rotational velocity of 100 rpm, the mean residence time is 4 min. The screw is composed of the following elements: ¼ D spacer—4 D conveyor elements (helix 3/2 L/D)—7 D conveyor elements (helix 1L/D)—1¾×D KB 1—2×D GFA (helix 1L/D)—2′/2×D KB2—10×D GFA (helix 1L/D)—4′/2×D KB3—2×D conveyor elements (helix 1L/D)—1×D distributive flow (1L/D)—2×D KB4 rev—1×D conveyor elements (helix 1L/D)—1×D KB 5—4×D conveyor elements (helix 1L/D). The kneading blocks are composed of individual kneading discs each having a width of ¼ or ⅛ D. Using a certain arrangement of the two different types (kneading disc 0° (S) and kneading disc 90° (F.)), it is possible to generate the angles 30°, 60°, 90°, 120° and 180°. Table 2 lists the angles generated by the respective disc (S or F).

TABLE 2
Composition of the kneading blocks KB1-KB5

| | |
|---|---|
| Kneading block 1 | S(0°)-F(30°)-F(60°)-S(90°)-F(90°)-S(90°)-S(60°) |
| Kneading block 2 | S(0°)-F(30°)-S(30°)-S(60°)-S(60°)-S(60°)-S(60°)- |
| | S(60°)-F(90°)-S(90°) |
| Kneading block 3 | S(0°)-F(30°)-S(30°)-S(60°)-F(90°)-S(90°) |
| Kneading block 4 | S(0°)-F(−30°)-F(−60°)-F(−60°)-F(−60°)-S(−90°) |
| Kneading block 5 | S(0°)-F(−30°)-S(30°)-1/8 F(30°)-1/8 S(30°) |

The swollen mass is extruded through an oval die and cut into pieces comprising the desired dose (height: 5 mm, width: 10 mm, length: 17 mm).

The content of a 1 g chewable is determined using a calculation based on the amount of powder components present. A chewable is composed of the following proportions of solids:

TABLE 3
Amount of ingredients per chewable (1.0 g) obtained by extrusion

| | mg |
|---|---|
| Praziquantel | 52.16 |
| Pig liver powder | 126.81 |
| Dicalcium phosphate | 50.70 |
| Croscarmellose-Sodium | 90.56 |
| Mannitol | 253.57 |

The liquid constituents of a chewable are, determined based on the amount of solid ingredients and the dosage of the swelling liquids, of the following composition:

TABLE 4
Liquid content of each chewable obtained by extrusion (1.0 g)

| | mg |
|---|---|
| Water | 142.07 |
| Hyaluronic acid | 0.0284 |
| Glycerol 100% | 284.13 |

The texture of the chewables is determined by texture profile analysis (TPA) according to Bourne and Szczesniak (Bourne, M. C. and Szczesniak, A. S. 2003, “Sensory evaluation I Texture”, Encyclopedia of Food Sciences and Nutrition (Second Edition), B. Caballero, Oxford, Academic Press, 5167-5174). A representation of the hardness profile determined in this manner of the chewables obtained by extrusion, a dog sausage and other commercially available soft chewable tablets is given in FIG. 3, FIG. 4 shows the hardness of the chewables obtained by extrusion in comparison to various foodstuffs and soft chewable tablets (“Soft Chews”). During a one-month storage in a sealed glass vessel, the chewables obtained by extrusion showed no subsequent hardening (FIG. 5).

The disintegration time is determined according to Ph. Eur. 8.0. Here, the disintegration time of the soft chewables obtained by extrusion as described in Example 4 is less than 30 min. When stored sealed (glass) at room temperature, the disintegration time remains unchanged for a month (FIG. 6).

The release from the chewables obtained by extrusion as described in Example 4 is tested in accordance with Ph. Eur. 8.0 and compared to the corresponding values of the comparative product “Milbemax® für Hunde” (FIG. 7). For these release analyses in 900 ml of water at 37° C. using a paddle stirrer apparatus, a UV probe was employed, the measurement taking place at a detected wavelength of 210 nm. It has to be noted that the release method did not incorporate a chewing step. Such a step would result in a higher increase of the concentration of the pharmaceutical during the first part of the release.

With a similarity factor of f₂=65.67 (European Medicine Agency 2011, EMA/CVMP/016/00-Rev.2), the release profile is similar to that of the approved product “Milbemax® für Hunde”.

For comparison, the following products were used:

Plerion®: Intervet, Vienna, Austria

Milbemax® für Hunde: Novartis Tiergesundheit GmbH, Munich, Germany

Dog sausage: DOGWURST, Vitakraft, Bremen, Germany; reference period: August 2014

Frolic® Mini: Mars GmbH, Verden, Germany; reference period: July 2013

### Examples 5 to 8

Using the process according to the invention, it is possible to prepare other chewables having different compositions analogously to Example 4 in a continuous process.

In contrast to Example 4, in all of Examples 5-8, the liquid F_{V} for preswelling is water instead of an aqueous hyaluronic acid solution (HA 1 mg/5 ml of water).

The compositions of the chewables are shown in Tables 5 and 6.

TABLE 5
Amount of the ingredients in the chewables (1.0 g each) of
Examples 5 and 6

| Example 5 | | Example 6 | |
|---|---|---|---|
| | mg per | | mg per |
| Component | chewable | Component | chewable |
| Imidacloprid | 100 | Moxidectin | 5 |
| Pig liver powder | 125 | Pig liver powder | 130 |
| Dicalcium phosphate | 40 | Dicalcium phosphate | 68 |
| Croscarmellose- | 83 | Croscarmellose- | 120 |
| Sodium | | Sodium | |
| Mannitol | 225 | Mannitol | 250 |
| Water | 142 | Water | 142 |
| Glycerol 100% | 285 | Glycerol 100% | 285 |

TABLE 6
Amount of the ingredients in the chewables (1.0 g) of Examples 7 and 8

| Example 7 | | Example 8 | |
|---|---|---|---|
| | mg per | | mg per |
| Component | chewable | Component | chewable |
| Enrofloxacin | 50 | (2-Chloro-N-(1-cyanocyclo- | 100 |
| | | propyl)-5-[2′-methyl-5′- | |
| | | (pentafluoroethyl)-4′-(tri- | |
| | | fluoromethyl)-2′H-1,3′-bi- | |
| | | pyrazol-4-yl]benzamide)* | |
| Pig liver powder | 125 | Pig liver powder | 100 |
| Dicalcium | 50 | Dicalcium phosphate | 40 |
| phosphate | | | |
| Croscarmellose- | 90 | Croscarmellose-Sodium | 83 |
| Sodium | | | |
| Mannitol | 258 | Mannitol | 250 |
| Water | 142 | Water | 142 |
| Glycerol 100% | 285 | Glycerol 100% | 285 |

*Example Ic-2 in WO2014/122083

### Preferred Embodiments of the Invention

The preferred embodiments of the invention are summarized below.

1. Process for preparing shaped articles for administration to animals, which comprises the following steps:

a) provision of a powder mixture of solid ingredients of the shaped articles,

b) mixing of the powder mixture obtained in step a) with at least one first liquid F_{V} having a volume F1, giving a preswollen mixture,

c) mixing of the preswollen mixture obtained in step b) with at least one second liquid F_{K} having a volume F2, where F2>F1, giving a swollen mixture,

d) forming of the swollen mixture obtained in step c) with formation of the shaped articles.

2. Process according to embodiment 1, where the ratio by volume of F2 to F1 is >1.1, preferably in the range from 1.2 to 5, more preferably in the range from 1.5 to 3.

3. Process according to embodiment 1 or 2, where the ratio by weight of the amount of the first liquid F_{V} to the amount of the solid ingredients in the powder mixture is in the range from 0.01 to 1, preferably from 0.1 to 0.8, more preferably from 0.3 to 0.7.

4. Process according to any of the above-mentioned embodiments 1 to 3, where the ratio by weight of the amount of the second liquid F_{K} to the amount of the solid ingredients in the powder mixture is in the range from 0.1 to 2, preferably from 0.2 to 1.5, more preferably from 0.6 to 1.3.

5. Process according to any of the above-mentioned embodiments 1 to 4, where the first liquid Fv is selected from the group consisting of water and mixtures of water and glycerol.

6. Process according to any of the above-mentioned embodiments 1 to 5, where the second liquid F_{K} is selected from the group consisting of water, glycerol and mixtures of water and glycerol.

7. Process according to any of the above-mentioned embodiments 1 to 6, where the liquids F_{V} and/or F_{K} contain one or more ingredients.

8. Process according to any of the above-mentioned embodiments 1 to 6, where the liquid F_{V} contains one or more ingredients.

9. Process according to any of the above-mentioned embodiments 7 or 8, where the ingredients added to the liquids are humectants.

10. Process according to embodiment 9, where the humectant is hyaluronic acid which preferably has a number-average molecular weight of 8000 to 15 000.

11. Process according to any of the above-mentioned embodiments 1 to 10, where the period of time between the addition of the preswelling liquid F_{V} in step b) and the swelling liquid F_{K} in step c) (preswelling time) is up to 300 s.

12. Process according to any of the above-mentioned embodiments 1 to 11, where the total time between the addition of the preswelling liquid F_{V} and the forming step d) is up to 80 minutes.

13. Process according to any of the above-mentioned embodiments 1 to 12, where a temperature of the mixtures of 50° C., preferably a temperature of the mixtures of 40° C., is not exceeded and the temperature of the mixtures is particularly preferably between 20 and 30° C.

14. Process according to any of the above-mentioned embodiments 1 to 13, where the process is carried out in a device selected from the group consisting of mixers, kneaders and extruders.

15. Process according to any of the above-mentioned embodiments 1 to 14, where steps b)-d) of the process are carried out continuously in an extruder.

16. Process according to any of the above-mentioned embodiments 1 to 15, where the forming in step d) comprises cutting of a strand.

17. Process according to any of the above-mentioned embodiments 1 to 16, where the process does not comprise a subsequent hardening or curing step of the shaped articles obtained after forming in step d) and these shaped articles can be packaged and/or stored protected from moisture immediately after step d).

18. Process according to any of the above-mentioned embodiments 1 to 17, where the ingredients are selected from the group consisting of:

a) one or more pharmaceutically active substances,

b) one or more flavourings,

c) one or more gel formers,

d) one or more fillers,

e) one or more liquids selected from the group of the liquids F_{V} and F_{K}

f) optionally one or more auxiliaries such as, for example, formulation auxiliaries, glidants, lubricants, disintegrants, humectants and preservatives.

19. Process according to any of the above-mentioned embodiments 1-18, where the ingredients contain one or more pharmaceutically active substances a) selected from the group consisting of oral veterinary pharmaceuticals such as antiparasitics, acaricides, insecticides, antimicrobial substances, antivirally active substances, antibiotics, antiphlogistics, psychotropic substances, proton pump inhibitors and anthelminthics, preferably praziquantel.

20. Process according to any of the above-mentioned embodiments 18 or 19, where the flavouring b) is selected from the group consisting of meat, poultry and fish flavourings of animal or synthetic origin, in particular dried meat, poultry and fish powders, especially pig liver powder.

21. Process according to any of the above-mentioned embodiments 18 to 20, where the gel former c) is selected from the group of compounds having a glycerol binding value (GBV) of more than 40 after 3 hours.

22. Process according to any of the above-mentioned embodiments 18 to 20, where the gel former c) is selected from the group of compounds having a glycerol binding value GBV of more than 60 after 3 hours.

23. Process according to any of the above-mentioned embodiments 18 to 22, where the gel former c) is selected from the group of compounds consisting of cellulose derivatives, polyacrylic acids, pectins, alginates, agar, carrageen and xanthan gum.

24. Process according to the above-mentioned embodiment 23, where the cellulose derivatives used as gel formers c) are selected from the group consisting of carboxymethylcellulose, hydroxyethylcellulose, hydroxypropylcellulose, methylcellulose and ethylcellulose.

25. Process according to any of the above-mentioned embodiments 23 and 24, where the cellulose derivative used as gel former c) is crosslinked carboxymethylcellulose (croscarmellose).

26. Process according to any of the above-mentioned embodiments 23 to 25, where the shaped article contains one or more cellulose derivatives and hyaluronic acid.

27. Process according to any of the above-mentioned embodiments 18 to 26, where the fillers d) are selected from the group consisting of solid sugar alcohols and inorganic calcium, magnesium, sodium and potassium salts.

28. Process according to embodiment 27, where the sugar alcohol used as filler d) is selected from the group consisting of compounds of the formula:

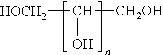

where n≥3.

29. Process according to embodiment 27, where the sugar alcohol used as filler d) is mannitol, xylitol or sorbitol.

30. Process according to any of the above-mentioned embodiments 18 to 27, where at least one of the fillers d) used is selected from the group consisting of lactose, cellulose, starch, sucrose and poorly soluble inorganic salts (solubility in 100 g of water at 20° C. of less than 1 g), preferably dicalcium phosphate dihydrate.

31. Process according to any of the above-mentioned embodiments 1 to 30, where the shaped articles contain less than 5% by weight starch or starch products.

32. Process according to any of the above-mentioned embodiments 1 to 31, where the shaped articles contain less than 5% by weight sucrose.

33. Shaped articles, obtainable according to any of the above-mentioned embodiments 1 to 32, containing:

a) one or more pharmaceutically active substances,

b) one or more flavourings,

c) one or more gel formers,

d) one or more fillers,

e) one or more liquids selected from the group of the liquids F_{V} and F_{K},

f) optionally one or more auxiliaries such as, for example, formulation auxiliaries such as glidants, lubricants, disintegrants, humectants and preservatives.

34. Shaped articles, obtainable according to any of the above-mentioned embodiments 1 to 32, containing:

0.1 to 30% by weight of the pharmaceutically active substances a),

1 to 25% by weight of the flavourings b),

1 to 20% by weight of the gel formers c),

5 to 60% by weight of the fillers d),

10 to 60% by weight of the liquids e),

0 to 30% by weight of one or more auxiliaries,

where the sum of the percentages is 100% by weight.

35. Shaped articles according to embodiment 33 or 34, characterized in that they have at least one of the parameters below:
a smooth surface,
a volume of up to 10 ml,
a disintegration time according to Ph. Eur. 8.0 of less than 30 minutes,
release according to Ph. Eur. 8.0 after 40 minutes of more than 50%,
moistness (loss on drying at 110° to a constant weight) of 3-30%
hardness determined by texture profile analysis (TPA) according to Bourne and Szczesniak between 2 and 15 N (preferably between 2 and 7 N).

36. Use of the shaped articles according to the above-mentioned embodiments 33 to 35 for administration to animals, preferably dogs and cats.

37. Use of the shaped articles according to the above-mentioned embodiments 33 to 35 for administration to animals, preferably dogs and cats, for treating helminthiases.

### DESCRIPTION OF THE FIGURES

FIG. 1: Processing time of the batchwise preparation of chewables according to the invention with and without additional preswelling

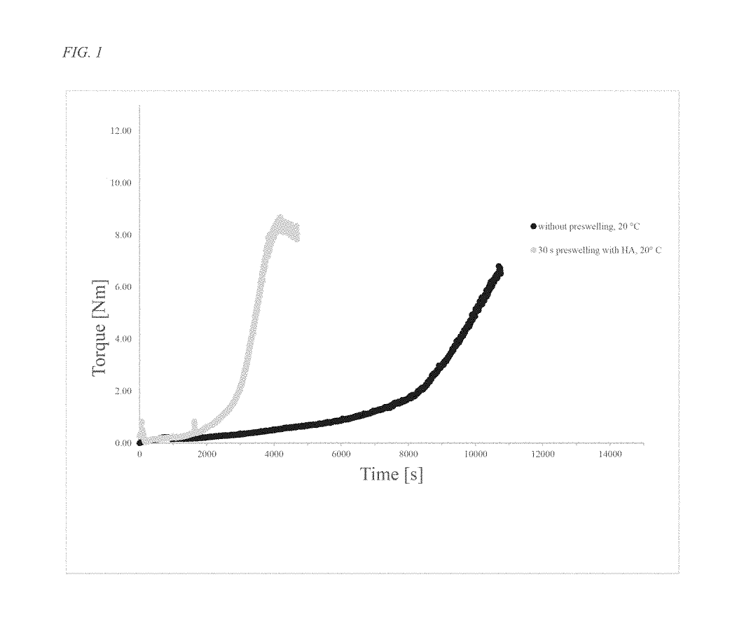

FIG. 2: Schematic drawing of the dosage units and the screw configuration in the extruder

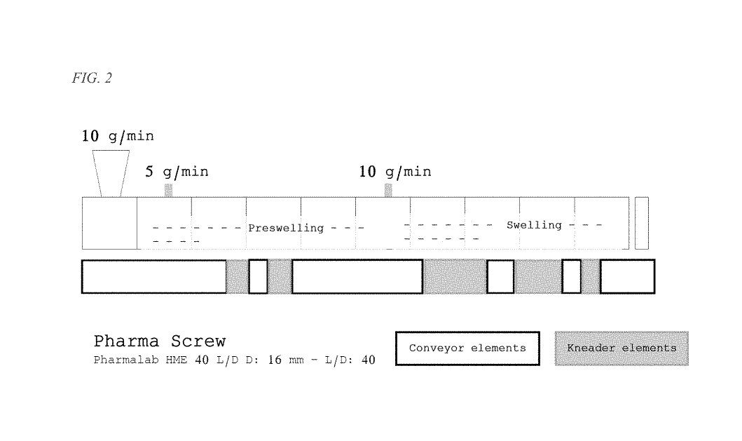

FIG. 3: Texture profiles of various “soft chews”, conventional meat sausage and a chewable according to the invention

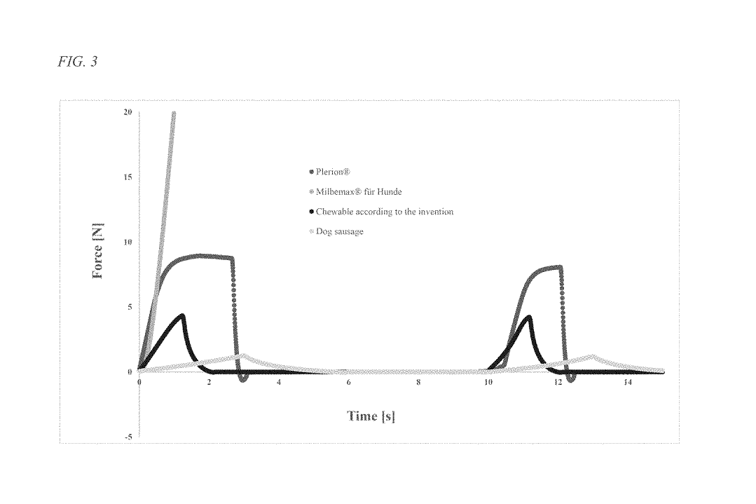

FIG. 4: Comparison of the hardness of different foodstuffs and chewables, mean±SD, n=6.

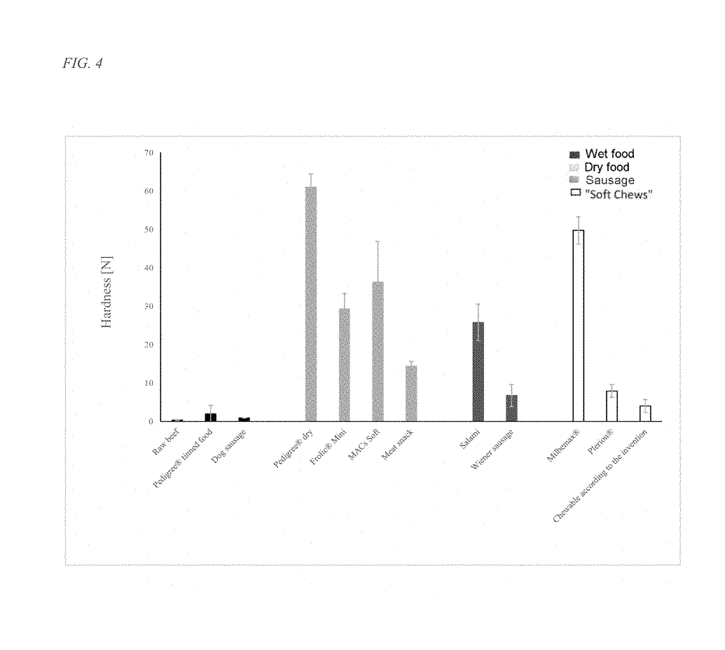

FIG. 5: Hardness of various chewables during storage

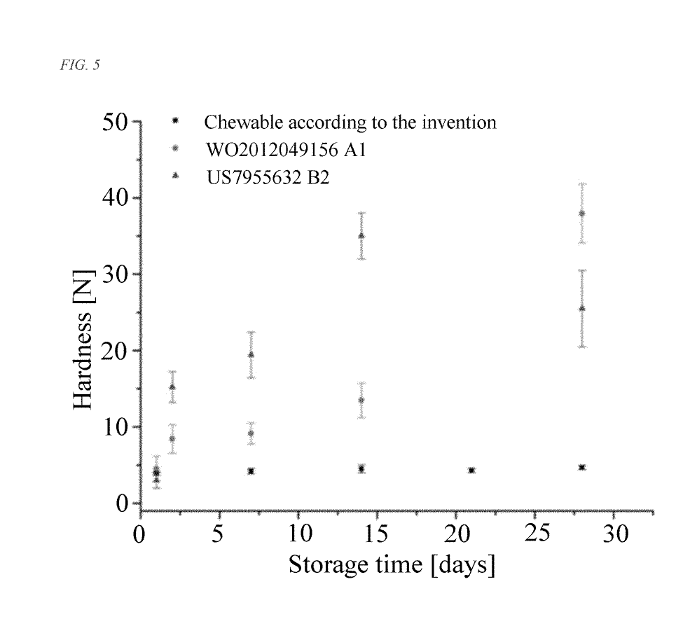

FIG. 6: Disintegration time of stored chewables according to the invention, n=3, mean±SD

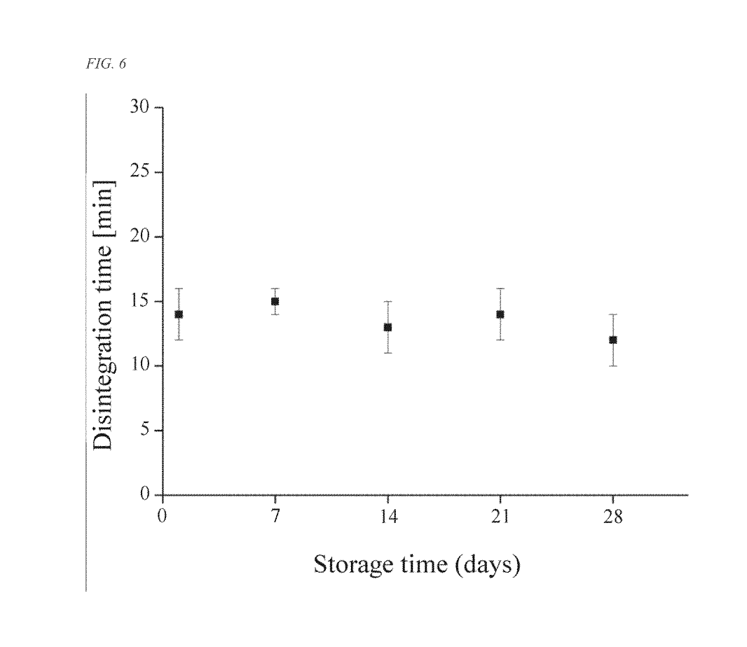

FIG. 7: Release of a soft chewable and the comparative product Milbemax® für Hunde, mean±SD, n=3.

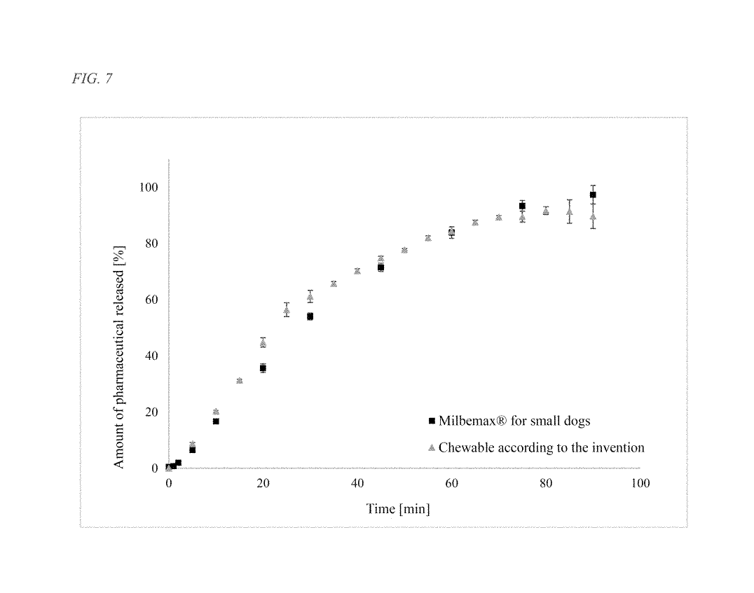

1. Process for preparing a shaped article for administration to an animal, which comprises:
a) providing a powder mixture of solid ingredients of the shaped article,
b) mixing the powder mixture obtained in a) with at least one first liquid F_{V} having a volume F1, giving a preswollen mixture,
c) mixing the preswollen mixture obtained in b) with at least one second liquid F_{K} having a volume F2, where F2>F1, giving a swollen mixture,
d) forming the swollen mixture obtained in c) with formation of the shaped article.

2. Process according to claim 1, where the ratio by volume of F2 to F1 is >1.1, optionally in the range from 1.2 to 5, optionally in the range from 1.5 to 3.

3. Process according to claim 1, where the ratio by weight of the amount of the first liquid F_{V} to the amount of the solid ingredients in the powder mixture is in the range from 0.01 to 1, optionally from 0.1 to 0.8, optionally from 0.3 to 0.7.

4. Process according to claim 1, where the ratio by weight of the amount of the second liquid F_{K} to the amount of the solid ingredients in the powder mixture is in the range from 0.1 to 2, optionally from 0.2 to 1.5, optionally from 0.6 to 1.3.

5. Process according to claim 1, where the first liquid F_{V} is selected from the group consisting of water and mixtures of water and glycerol.

6. Process according to claim 1, where the second liquid F_{K} is selected from the group consisting of water, glycerol and mixtures of water and glycerol.

7. Process according to claim 1, where the period of time between addition of the preswelling liquid F_{V} in step b) and the swelling liquid F_{K} in c) (preswelling time) is up to 300 s.

8. Process according to claim 1, where the ingredients are selected from the group consisting of:
a) one or more pharmaceutically active substances,
b) one or more flavourings,
c) one or more gel formers,
d) one or more fillers,
e) one or more liquids selected from the group of the liquids F_{V} and F_{K},
f) optionally one or more auxiliaries optionally, formulation auxiliaries, glidants, lubricants, disintegrants, humectants and preservatives.

9. Process according to claim 1, where the ingredients comprise one or more pharmaceutically active substances a) selected from the group consisting of oral veterinary pharmaceuticals optionally antiparasitics, acaricides, insecticides, antimicrobial substances, antibiotics and anthelminthics, optionally praziquantel.

10. Process according to claim 8, where the gel former c) is selected from the group of the compounds having a glycerol binding value (GBV) of more than 40 after 3 hours.

11. Process according to claim 8, where the gel former c) is selected from the group of compounds consisting of cellulose derivatives, polyacrylic acids, pectins, alginates, agar, carrageen and xanthan gum.

12. Process according to claim 8, where the fillers d) are selected from the group consisting of solid sugar alcohols and inorganic calcium, magnesium, sodium and potassium salts.

13. Process according to claim 1, where the process is carried out in an extruder.

14. Shaped article, obtainable according to claim 1, comprising:
a) one or more pharmaceutically active substances,
b) one or more flavourings,
c) one or more gel formers,
d) one or more fillers,
e) one or more liquids selected from the group of the liquids F_{V} and F_{K},
f) optionally one or more auxiliaries optionally, for example, formulation auxiliaries such as glidants, lubricants, disintegrants, humectants and preservatives.

15. Shaped article according to claim 14, having at least one of the parameters below:
a smooth surface,
a volume of up to 10 ml,
a disintegration time according to Ph. Eur. 8.0 of less than 30 minutes,
release according to Ph. Eur. 8.0 after 40 minutes of more than 50%,
moistness (loss on drying at 110° to a constant weight) of 3-30%
hardness determined by texture profile analysis (TPA) according to Bourne and Szczesniak between 2 and 15 N (optionally between 2 and 7 N).

16. A product comprising a shaped article according to claim 14 for administration to one or more animals, optionally dogs and cats, optionally for treating helminthiases.
